(19) United States
(12) Patent Application Publication
(10) Pub. No.: US 2015/0342899 A1
(43) Pub. Date: Dec. 3, 2015

(54) COMPOSITIONS AND METHODS FOR TRANSDERMAL DELIVERY OF AMPHETAMINE AND CLONIDINE

(71) Applicant: NOVEN PHARMACEUTICALS, INC., Miami, FL (US)

(72) Inventors: Joshua KULAKOFSKY, Miami, FL (US); Puchun LIU, Miami, FL (US)

(73) Assignee: Noven Pharmaceuticals, Inc., Miami, FL (US)

(21) Appl. No.: 14/654,919

(22) PCT Filed: Dec. 23, 2013
(86) PCT No.: PCT/US2013/077401

§ 371 Date: Jun. 23, 2015

Related U.S. Application Data
(60) Provisional application No. 61/746,977, filed on Dec. 28, 2012.

(51) Int. Cl. A61K 9/70; A61K 31/4168; A61K 31/137

(52) U.S. Cl. CPC A61K 9/7061; A61K 31/137; A61K 31/4168

(57) ABSTRACT

Described are compositions and methods for combination therapy using a stimulant therapeutically active agent (such as amphetamine) and a non-stimulant therapeutically active agent (such as clonidine), such as for the treatment of Attention Deficit Disorder (ADD) and/or Attention Deficit/Hyperactivity Disorder (ADHD). Methods of making and using such systems also are described.

### CROSS-REFERENCE TO RELATED APPLICATIONS

This application claims the benefit under 35 USC §119(e) to U.S. provisional application 61/746,977, filed Dec. 28, 2012, the entire contents of which are incorporated herein by reference in their entirety.

### FIELD

Described herein are compositions and methods for combination therapy using a stimulant therapeutically active agent (such as amphetamine) and a non-stimulant therapeutically active agent (such as clonidine), such as for the treatment of Attention Deficit Disorder (ADD) and/or Attention Deficit/Hyperactivity Disorder (ADHD). In specific embodiments, the compositions are transdermal drug delivery systems comprising the active agents and the methods comprise transdermal administration of the active agents.

### BACKGROUND

Attention Deficit Disorder (ADD) and Attention Deficit/Hyperactivity Disorder (ADHD) (severally and collectively hereinafter referred to as “ADHD”) are considered to be developmental disorders of self-control. They present as problems with attention span, impulse control and activity level. These problems are reflected in impairment of a person's will or capacity to control his or her own behavior relative to the passage of time and to keep future goals and consequences in mind.

Methylphenidate has been used to treat ADD/ADHD in both children and adults, and is available in transdermal (Daytrana®) and oral (e.g., Ritalin®) dosage forms. Amphetamine has been used to treat ADD/ADHD and currently is commercially available in oral dosage forms (e.g., Adderall®). Both methylphenidate and amphetamine are central nervous system stimulants.

Clonidine has been proposed for the treatment of ADD/ADHD. Clonidine currently is commercially available in ophthalmic, oral, transdermal (Catapres-TTS®), and injectable forms. Catapres-TTS® is approved for the treatment of hypertension.

Transdermal delivery systems (adhesive patches) as dosage forms have been the subject of a vast number of patent applications over the last 25 years, yielding many patents but few commercial products in comparison. To those working in the field, the relatively small number of commercial products is not surprising. Although regulatory, economic, and market hurdles play a role in limiting the number of products on the market, the task of developing a transdermal delivery system that achieves desired physical and pharmacokinetic parameters to satisfy physician and patient demand is more daunting. Parameters to be considered during commercial product development may include drug solubility, drug stability (e.g., as may arise from interaction with other component materials and/or the environment), delivery of a therapeutic amount of drug at a desired delivery rate over the intended duration of use, adequate adhesion at the anatomical site of application, integrity (e.g., minimal curling, wrinkling, delaminating and slippage) with minimal discomfort, irritation and sensitization both during use and during and after removal, and minimal residual adhesive (or other components) after removal. Size also may be important from a manufacturing and patient viewpoint, and appearance may be important from a patient viewpoint. The physical manufacturing and production aspects of commercial product development (e.g., the identity and costs of materials, equipment, and labor) and supporting analytical methods required for regulatory compliance also can be significant.

There remains a need for effective methods of treating ADHD, and for effective transdermal drug delivery systems useful for treating ADHD.

### SUMMARY

In accordance with some embodiments, there are provided transdermal drug delivery systems for the treatment of ADD and/or ADHD in the form of a flexible finite system for topical application, comprising a polymer matrix comprising a stimulant therapeutically active agent, such as a central nervous system stimulant, e.g., amphetamine or methylphenidate, and a non-stimulant therapeutically active agent, such as an alpha-2 agonist, e.g., clonidine or guanfacine.

In some embodiments, the polymer matrix comprises an acrylic polymer, which optionally may be a non-acid functional acrylic polymer. In specific embodiments, the polymer matrix comprises amphetamine, clonidine and a non acid-functional acrylic polymer. In some embodiments, the polymer matrix comprises a first non-acid functional acrylic polymer and a second non-acid functional acrylic polymer. In accordance with any embodiments, the polymer matrix may comprise a non acid-functional acrylic polymer that includes methacrylate monomers and 2-ethylhexyl acrylate monomers. In accordance with any embodiments, the polymer matrix may comprise non acid-functional acrylic polymer that includes methacrylate monomers, 2-ethylhexyl acrylate monomers, and amide-group containing monomers.

In accordance with any embodiments, the transdermal drug delivery system may comprise a backing layer and/or a release liner.

In accordance with any embodiments, the transdermal drug delivery system may be effective to achieve different pharamacokinetic profiles for the active agents formulated therein, such as achieving one pharamacokinetic profile for the stimulant therapeutically active agent (e.g., amphetamine or methylphenidate) and a different pharamacokinetic profile for the non-stimulant therapeutically active agent (e.g., clonidine or guanfacine). In specific embodiments, the stimulant therapeutically active agent (e.g., amphetamine or methylphenidate) is delivered at a faster rate and/or over a relatively short period of time and/or exhibits immediate release while the non-stimulant therapeutically active agent (e.g., clonidine or guanfacine) is delivered at a more gradual rate and/or over a relatively extended period of time and/or exhibits sustained release.

Also provided are methods of treating ADD/ADHD comprising applying a transdermal drug delivery system as described herein to the skin or mucosa of a subject in need thereof. In some embodiments, the subject is a human subject. In some embodiments, the transdermal drug delivery system is applied for a duration of up to 24 hours. In some embodiments, the methods result in different pharamacokinetic profiles for the active agents formulated therein, such as achieving one pharamacokinetic profile for the stimulant therapeutically active agent (e.g., amphetamine or methylphenidate) and a different pharamacokinetic profile for the non-stimulant therapeutically active agent (e.g., clonidine or guanfacine). In specific embodiments, the stimulant therapeutically active agent (e.g., amphetamine or methylphenidate) is delivered at a faster rate and/or over a relatively short period of time and/or exhibits immediate release while the non-stimulant therapeutically active agent (e.g., clonidine or guanfacine) is delivered at a more gradual rate and/or over a relatively extended period of time and/or exhibits sustained release.

Also provided are transdermal drug delivery systems as described herein, for use in treating ADD or ADHD.

Also provided are uses of a stimulant therapeutically active agent and a non-stimulant therapeutically active agent in the preparation of a medicament for treating ADD or ADHD wherein the medicament is in the form of a flexible finite system for topical application, comprising a polymer matrix comprising the stimulant therapeutically active agent and the non-stimulant therapeutically active agent.

Also provided are methods of preparing a transdermal drug delivery system as described herein, comprising preparing a polymer matrix comprising a stimulant therapeutically active agent and a non-stimulant therapeutically active agent.

### BRIEF DESCRIPTION OF THE DRAWINGS

FIG. 1 illustrates the flux of amphetamine (▴) and clonidine (♦) (μg/cm²/hr) over time (0-24 hours) from a transdermal delivery system according to the invention.

### DETAILED DESCRIPTION

Described herein are compositions and methods for the combination therapy of Attention Deficit Disorder (ADD) and Attention Deficit/Hyperactivity Disorder (ADHD) (severally and collectively, “ADHD”) using a stimulant therapeutically active agent (such as amphetamine) and a non-stimulant therapeutically active agent (such as clonidine). Also described are transdermal drug delivery systems comprising the active agents, methods comprising the transdermal administration of the active agents, and methods of making the transdermal drug delivery systems.

The inventors surprisingly discovered that the combination of a stimulant therapeutically active agent (such as amphetamine) and a non-stimulant therapeutically active agent (such as clonidine) achieves synergistic effects in the treatment of ADHD. For example, by using a combination of a stimulant therapeutically active agent (such as amphetamine) and a non-stimulant therapeutically active agent (such as clonidine), one or both of the active agents can be administered at a dose lower than is required for a therapeutic effect if the drug is used alone, while still achieving a therapeutic effect. In some embodiments, one or both of the drugs can be administered at a dose lower than its/their threshold dose for inducing side effects. These characteristics of the invention are particularly advantageous because therapeutic effect can be achieved with less of each drug and, in some embodiments, with reduced or minimized side effects.

In accordance with some aspects, there are provided transdermal drug delivery systems and methods for the transdermal delivery of a stimulant therapeutically active agent (such as amphetamine) and a non-stimulant therapeutically active agent (such as clonidine). In some embodiments, the systems have a lower amount of one or both drugs per unit area than would be required for a therapeutic effect if the drug were formulated in a comparable system with only the one drug. In some embodiments, the systems are formulated with an amount of one or both drugs that is less than the threshold amount for inducing side effects.

In accordance with some aspects, the transdermal drug delivery system is effective to achieve different pharamacokinetic profiles for the active agents formulated therein, such as achieving one pharamacokinetic profile for the stimulant therapeutically active agent (e.g., amphetamine or methylphenidate) and a different pharamacokinetic profile for the non-stimulant therapeutically active agent (e.g., clonidine or guanfacine), such as one active agent being delivered at a faster rate and/or over a relatively short period of time as compared to another and/or one active agent exhibiting immediate release and another exhibiting a more sustained release. In further specific aspects, the stimulant therapeutically active agent (e.g., amphetamine or methylphenidate) is delivered at a faster rate and/or over a relatively short period of time and/or exhibits immediate release, while the non-stimulant therapeutically active agent (e.g., clonidine or guanfacine) is delivered at a slower rate and/or over an extended period of time and/or exhibits a more sustained release, or vice versa.

### DEFINITIONS

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein generally means that the described composition (e.g., transdermal drug delivery system, polymer matrix, etc.) comprises less than about 5%, less than about 3%, or less than about 1% by weight, based on the total weight of the composition at issue, of the excluded component.

As used herein “subject” denotes any animal in need of drug therapy, including humans. For example, a subject may be suffering from ADHD.

As used herein, the phrases “therapeutically effective amount” and “therapeutic level” mean that drug dosage or plasma concentration in a subject, respectively, that provides the specific pharmacological response for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein, “active surface area” means the surface area of the drug-containing layer of the transdermal drug delivery system.

As used herein, “flux” (also called “permeation rate”) is defined as the absorption of a drug through skin or mucosal tissue, and is described by Fick's first law of diffusion:

J=−D (dCm/dx)

where J is the flux in g/cm²/sec, D is the diffusion coefficient of the drug through the skin or mucosa in cm²/sec and dCm/dx is the concentration gradient of the drug across the skin or mucosa.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system (e.g., a device) comprising a composition that releases therapeutically active agent upon application to the skin (or any other surface noted above). A transdermal drug delivery system may comprise a drug-containing layer, and, optionally, a backing layer and/or a release liner layer. In some embodiments, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. As described below, in one embodiment, the transdermal drug delivery system comprises a drug-containing polymer matrix that comprises a pressure-sensitive adhesive or bioadhesive, and is adopted for direct application to a user's (e.g., a subject's) skin. In other embodiments, the polymer matrix is non-adhesive and may be provided with separate adhesion means (such as a separate adhesive layer) for application and adherence to the user's skin.

As used herein, “polymer matrix” refers to a polymer composition which contains one or more drugs. In some embodiments, the matrix comprises a pressure-sensitive adhesive polymer or a bioadhesive polymer. In other embodiments, the matrix does not comprise a pressure-sensitive adhesive or bioadhesive. As used herein, a polymer is an “adhesive” if it has the properties of an adhesive per se, or if it functions as an adhesive by the addition of tackifiers, plasticizers, crosslinking agents or other additives. Thus, in some embodiments, the polymer matrix comprises a pressure-sensitive adhesive polymer or a bioadhesive polymer, with the therapeutically active agents dissolved or dispersed therein. The polymer matrix also may comprise tackifiers, plasticizers, crosslinking agents, enhancers, co-solvents, fillers, antioxidants, solubilizers, crystallization inhibitors, or other additives described herein. U.S. Pat. No. 6,024,976 describes polymer blends that are useful in accordance with the transdermal systems described herein. The entire contents of U.S. Pat. No. 6,024,976 is incorporated herein by reference.

As used herein, the term “pressure-sensitive adhesive” refers to a viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

In some embodiments, the polymer matrix is a pressure-sensitive adhesive at room temperature and has other desirable characteristics for adhesives used in the transdermal drug delivery art. Such characteristics include good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

As used herein, the term “rubber-based pressure-sensitive adhesive” refers to a viscoelastic material which has the properties of a pressure-sensitive adhesive and which contains at least one natural or synthetic elastomeric polymer.

In some embodiments, the transdermal drug delivery system includes one or more additional layers, such as one or more additional polymer matrix layers, or one or more adhesive layers that adhere the transdermal drug delivery system to the user's skin. In other embodiments, the transdermal drug delivery system is monolithic, meaning that it comprises a single polymer matrix layer comprising a pressure-sensitive adhesive or bioadhesive with drug dissolved or dispersed therein, and no rate-controlling membrane.

The transdermal drug delivery system also may include a drug impermeable backing layer or film. In some embodiments, the backing layer is adjacent one face of the polymer matrix layer. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotch Pak™ 1012 or 9732 backing material (a polyester film with an ethylene vinyl acetate copolymer heat seal layer) is useful in the transdermal drug delivery systems described herein.

The transdermal drug delivery system also may include a release liner, typically located adjacent the opposite face of the system as compared to the backing layer. When present, the release liner is removed from the system prior to use to expose the polymer matrix layer and/or an adhesive layer prior to topical application. Materials suitable for use as release liners are well-known known in the art and include the commercially available products of Dow Corning Corporation designated Bio-Release® liner and Syl-Off® 7610 (both silicone-based) and 3M's 1020, 1022, 9741, 9744, 9748, 9749 and 9755 Scotchpak™ (fluoropolymer coated polyester films).

The transdermal drug delivery system may be packaged or provided in a package, such as a pouchstock material used in the prior art for transdermal drug delivery systems. For example, DuPont's Surlyn® can be used in a pouchstock material.

As used herein, a “monolithic” transdermal drug delivery system may include a backing layer and/or release liner, and may be provided in a package.

Therapeutically Active Agents

As used herein “stimulant therapeutically active agent” includes central nervous system stimulants, including central nervous system stimulants currently used to treat ADD/ADHD, such as amphetamine and methylphenidate, and pharmaceutically acceptable salts thereof.

Amphetamine (alpha-methylphenethylamine) is a chiral drug. The commercially available oral amphetamine product Adderall® includes several different amphetamine salts, including amphetamine sulfate, amphetamine saccharate, and amphetamine aspartate monohydrate, in an overall ratio of d-amphetamine to 1-amphetamine of 3:1.

The compositions described herein may be formulated with amphetamine free base or any salt of amphetamine, or any prodrug thereof, or any combinations thereof, and with any isomeric content, and any combinations thereof. In specific embodiments, the compositions comprise d-amphetamine. In further specific embodiments the amphetamine component consists essentially of d-amphetamine (e.g., it contains no more than trace amounts of other amphetamine species). In still further specific embodiments the amphetamine component consists of d-amphetamine. In other specific embodiments, the composition comprises a prodrug of d-amphetamine, such as lisdexamfetamine, in the free base or any salt form, such as lisdexamfetamine dimesylate.

In addition to the salts mentioned above, exemplary suitable pharmaceutically acceptable salts of amphetamine are salts of weak inorganic and organic acids, and quaternary ammonium salts. These include without limitation, salts with acids such as sulfuric, phosphoric, hydrochloric, hydrobromic, hydriodic, sulfamic, citric, lactic, maleic, malic, succinic, tartaric, cinnamic, acetic, benzoic, gluconic, or ascorbic acid, or quaternary ammonium salts with organic esters of sulfuric, hydrohalic, or aromatic sulfonic acids, such as methyl chloride, methyl bromide, ethyl chloride, propyl chloride, butyl chloride, isobutyl chloride, benzylchloride, benzyl bromide, phenethyl bromide, naphthymethyl chloride, dimethyl sulfate, methyl benzenesulfonate, ethyl toluenesulfonate, ethylene chlorohydrin, propylene chlorobydrin, allyl bromide, methylallyl bromide or crotyl bromide esters.

As used herein “non-stimulant therapeutically active agent” includes therapeutically active agents that are not central nervous system stimulants, that may be useful to treat ADHD. In some embodiments, the non-stimulant therapeutically active agent is an alpha-2 agonist, such as guanfacine and clonidine. As with amphetamine, the non-stimulant therapeutically active agent may be present in its free base form and/or any pharmaceutically acceptable salt.

The concentration by weight of the therapeutically active agents in the transdermal drug delivery system is typically about 0.1 to about 50%, including about 0.1 to about 40% and about 0.3 to about 30%, such as 0.1 to 50%, 0.1 to 40% and 0.3 to 30%, all based on the total weight of the polymer matrix.

In some embodiments, the stimulant therapeutically active agent is amphetamine, and is present at an amount of from about 1% to about 50%, including from about 5% to about 40%, such as from about 10% to about 20% by weight, based on the total dry weight of the polymer matrix. In specific embodiments, the polymer matrix comprises about 15% by weight amphetamine, based on the total dry weight of the polymer matrix. In other specific embodiments, the polymer matrix comprises about 10% by weight amphetamine, based on the total dry weight of the polymer matrix. In other specific embodiments, the polymer matrix comprises about 20% by weight amphetamine, based on the total dry weight of the polymer matrix.

In some embodiments, the non-stimulant therapeutically active agent is clonidine, and is present at an amount from about 0.1% to about 50%, including from about 1% to about 20%, such as from about 1% to about 10% by weight, such as about 1, about 2, about 3, about 4, about 5, about 6, about 7, about 8, about 9 or about 10% by weight, based on the total dry weight of the polymer matrix. In specific embodiments, the polymer matrix comprises about 3, about 4, about 5, about 6, or about 7% by weight clonidine, based on the total dry weight of the polymer matrix.

The amount of therapeutically active agents may vary depending on the time span for which the system is to provide therapy. For most drugs, the passage of the drugs through the skin will be the rate-limiting step in delivery. A minimum amount of drug in the system is selected based on the amount of drug which passes through the skin in the time span for which the system is to provide therapy. In some embodiments, a system for the transdermal delivery as described herein is designed for daily use, e.g., is used for a period of time such as 8 hours, 10 hours, 12 hours, up to about 24 hours, or longer. Thus, in one embodiment, the systems comprise an amount of the therapeutically active agents sufficient to deliver therapeutically effective amounts of drug over a period of time of 8 hours, 10 hours, 12 hours, up to about 24 hours, or longer.

Regardless of whether there is high-loading or low-loading of the therapeutically active agents into the transdermal drug delivery system, the pressure-sensitive adhesive composition can be formulated to maintain acceptable shear, tack, and peel adhesive properties, by selecting suitable polymer components and, optionally, other components.

Acrylic Polymers

The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids. In some embodiments, the acrylic polymer constitutes from about 2% to about 95% by weight of the polymer content of the polymer matrix, including about 3% to about 90% and about 5% to about 85%, such as 2% to 95%, 3% to 90% and 5% to 85%. In some embodiments, the amount and type of acrylic polymer is dependent on the type and amount of therapeutically active agents used.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups.

Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups.

In accordance with specific embodiments, the polymer matrix comprises or consists of one or more non acid-functional acrylic polymers as the polymer component. Non acid-functional acrylic polymers include those formed from acrylic esters copolymerized with other monomers that do not include acid-functional groups. Non acid-functional acrylic polymers include homopolymers, copolymers, terpolymers, etc., of acrylic acids and esters. As used herein, “non acid-functional acrylic polymer” includes polymers that include monomers that have one or more amide groups. In specific embodiments, the non acid-functional acrylic polymer includes methacrylate monomers and 2-ethylhexyl acrylate monomers. In specific embodiments the non acid-functional acrylic polymer includes methacrylate monomers, 2-ethylhexyl acrylate monomers, and amide-group containing monomers.

When the acrylic polymer component includes more than one non acid-functional acrylic polymer, the polymers can be present in any ratio that results in a product with satisfactory physical and pharmacokinetic properties. For example, the acrylic polymer component can include from 0-100% of a first non acid-functional acrylic polymer and from 100-0% of a second non acid-functional acrylic polymer, based on the total dry weight of the acrylic component, including about 10 to about 90%, about 15- about 85%, about 20 to about 80%, about 25 to about 75%, about 33 to about 66%, and about 50% of the first non acid-functional acrylic polymer, and the balance being the second (or third, etc.) non acid-functional acrylic polymer(s). In specific embodiments, the acrylic polymer component includes about 80% of a first non acid-functional acrylic polymer and about 20% of a second non acid-functional acrylic polymer, based on the total polymer content.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, N.Y. (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

Suitable acrylic polymers also include pressure-sensitive adhesives which are commercially available, such as the acrylic-based adhesives sold under the trademarks DURO-TAK® by National Starch and Chemical Corporation, Bridgewater, N.J. (such as DURO-TAK® 87-900A, 87-2287, -4098, -2852, -2196, -2296, -2194, -2516, -2070, -2353, -2154, -2510, -9085, -9088 and 73-9301). Other suitable acrylic adhesives include those sold under the trademark EUDRAGIT® by Roehm Pharma GmbH, Darmstadt, Germany, those sold by Cytec Surface Specialties, St. Louis, Mo., under the trademarks GELVA® Multipolymer Solution (such as GELVA® 3087, 2480, 788, 737, 263, 1430, 1753, 1151, 2450, 2495, 3067, 3071, and 3235). Non-limiting commercial examples of functional adhesives with a reactive functional hydroxyl group in the polymeric chain Mein& GELVA® 737, 788, and 1151, and DURO-TAK® 87-2287, -4287, -2510 and -2516, Suitable non acid-functional acrylic polymers which are commercially available include those sold by Henkel (Dusseldorf, Germany), under the Duro-Tak® brand such as Duro-Tak 900A, and those sold by Monsanto (St. Louis, Mo.) under Gelva® Multipolymer Solution brand, such as Gelva 3087 and Gelva-3235.

In some embodiments, the acrylic polymer(s) constitute(s) up to 100% by weight of the polymer content of the polymer matrix. Other optional polymer components are discussed below. In embodiments using one or more such other polymer components, the acrylic polymer(s) may constitute from 0% to 99.9% by weight of the polymer content of the polymer matrix.

Silicon Polymers

In some embodiments, the polymer matrix comprises a silicone polymer. The term “silicone-based” polymer is used interchangeably with the terms silicon polymers, siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, N.Y. (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series, such as -4502, and -4600 series.

Further details and examples of silicone pressure-sensitive adhesives which are useful in the polymer matrices and compositions and methods described herein are mentioned in the following U.S. Pat. Nos. 4,591,622; 4,584,355; 4,585,836; and 4,655,767, which are all expressly incorporated by reference herein in their entireties. It should also be understood that silicone fluids are also contemplated for use in the polymer matrices and methods described herein.

In some embodiments, the polysiloxane, if present, constitutes from about 1 to about 99% of the polymer content of the polymer matrix, such as from about 9% to about 97% of the polymer content of the polymer matrix, including about 8% to about 97% and about 14% to about 94%, such as 9% to 97%, 8% to 97%, and 14% to 94%.

Soluble PVP

In some embodiments, the polymer matrix includes soluble PVP. Soluble PVP has been found to be highly effective in preventing crystallization of drugs in adhesive-type transdermal drug delivery system. Soluble PVP also may modulate the transdermal permeation rate of the drug.

The term “PVP or “polyvinylpyrrolidone” refers to a polymer, either a homopolymer or copolymer, containing N-vinylpyrrolidone as the monomeric unit. Typical PVP polymers are homopolymeric PVPs and the copolymer vinyl acetate vinylpyrrolidone. The homopolymeric PVPs are known to the pharmaceutical industry under a variety of designations including Povidone, Polyvidone, Polyvidonum, Polyvidonum soluble, and Poly(1-vinyl-2-pyrrolidone). The copolymer vinyl acetate vinylpyrrolidone is known to the pharmaceutical industry as Copolyvidon, Copolyvidone, and Copolyvidonum. The term “soluble” when used with reference to PVP means that the polymer is soluble in water and generally is not substantially cross-linked, and has a molecular weight of less than about 2,000,000. See, generally, Buhler, KOLLIDON®: POLYVINYLPRYRROLIDONE FOR THE PHARMACEUTICAL INDUSTRY, BASF Aktiengesellschaft (1992).

The amount and type of soluble PVP used may depend on the quantity and type of therapeutically active agents present, as well as the type of adhesive, but can be readily determined through routine experimentation. Typically, the PVP, if present, is present in an amount from about 1% to about 20% by weight, such as from about 5% to about 15% by weight, based on the total weight of the polymer matrix. However, the amount of PVP can be higher than 20% for example, up to 40%, depending on the particular drug used and on the desired properties of the blend.

The soluble PVP may have a molecular weight of about 2,000 to 1,100,000, including 5,000 to 100,000, and 7,000 to 54,000. In some embodiments, the soluble PVP has a molecular weight of from about 17,000 to about 90,000, such as from about 17,000 to about 60,000, including from 17,000 to 90,000 and from 17,000 to 60,000.

In some embodiments, the polymer matrix comprises a soluble PVP with a rubber-based pressure-sensitive adhesive and a polyacrylate polymer, such as a blend of an acrylic polymer, a polysiloxane and a soluble PVP.

In some embodiments, the polymer blend is chosen to affect the rate of drug delivery. More specifically, a plurality of polymers which may have different solubility parameters for the drug and which may be immiscible with each other, may be selected to adjust the solubility of the drug in the polymer matrix, thereby controlling the maximum concentration of the drug in the system, and modulating drug delivery through the dermis. These embodiment include blends of two or more acrylic polymers (including blends of two or more non-acid functional acrylic polymers), or blends of two or more polymers selected from acrylic polymers, silicone polymers and PVP polymers, or other polymers. The amount and type of each polymer can be adjusted so as to modify the saturation concentration of the drug in the polymer matrix in order to affect the rate of delivery of the drug from the system and through the skin.

Other Components

In some embodiments, the polymer matrix comprises a penetration enhancer. A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, the polymer matrix does not comprise a penetration enhancer.

When present, a penetration enhancer typically is used in an amount up to about 30% by dry weight of the polymer matrix, including up to 30% by weight, up to about 20% by weight, including 20% by weight, or up to about 10% by weight, up to 10% by weight, or up to 5% by weight, including up to 5% by weight, based on the dry weight of the polymer matrix.

The polymer matrix may further comprise various thickeners, fillers, and other additives or components known for use in transdermal drug delivery systems.

As noted above, in embodiments where the polymer matrix comprises a pressure-sensitive adhesive or bioadhesive, the polymer matrix can serve as an adhesive portion of the system (e.g., a reservoir device), or can serve as one or more layers of a multi-layer system. Alternatively, a polymer matrix comprising a pressure-sensitive adhesive or bioadhesive with drug dissolved or dispersed therein can constitute a monolithic device. In embodiments where the polymer matrix does not comprise an adhesive, but instead, for example, comprises a polymeric drug reservoir, it can be used in combination with one or more adhesive layers, or with a surrounding adhesive portion, as is well known to those skilled in the art.

In some embodiments, the system consists essentially of the polymer matrix layer. By “consists essentially of the polymer matrix layer” means that the system does not contain any other layers that affect drug delivery, such as an additional rate-controlling polymer layer, rate-controlling membrane, or drug reservoir layer. It will be understood, however, that the system that consists essentially of the polymer matrix layer may comprise a backing layer and/or release liner.

The system may be of any shape or size suitable for transdermal application.

The polymer matrices described herein may be prepared by methods known in the art. The polymer matrices can be formed into systems by methods known in the art. For example, the polymer matrix material can be applied to a backing layer and release liner by methods known in the art, and formed into sizes and shapes suitable for use. For example, after the polymer matrix is formed, it may be brought into contact with a support layer, such a releaser liner layer or backing layer, in any manner known to those of skill in the art. Such techniques include calender coating, hot melt coating, solution coating, etc.

For example, a polymer matrix can be prepared by blending the components of the polymer matrix, applying the matrix material to a support layer such as a backing layer or release liner, and removing any remaining solvents. The therapeutically active agents can be added at any stage. In one embodiment, all polymer matrix components, including the therapeutically active agents, are blended together. In another embodiment, the polymer matrix components other than the therapeutically active agents are blended together, and then the therapeutically active agents are dissolved or dispersed therein. The order of steps, amount of ingredients, and the amount and time of agitation or mixing can be determined and optimized by the skilled practitioner. An exemplary general method is as follows:

Appropriate amounts of polymer(s), enhancer(s), and organic solvent(s) (for example toluene, or ethyl acetate an/or isopropyl alcohol) are combined and thoroughly mixed together in a vessel, along with the active agents.

The formulation is then transferred to a coating operation where it is coated onto a protective release liner at a controlled specified thickness. The coated product is then passed through an oven in order to drive off all volatile processing solvents.

The dried product on the release liner is then joined to the backing material and wound into rolls for storage.

Appropriate size and shape “systems” are die-cut from the roll material and then pouched.

Other manufacturing methods are known in the art that are suitable for making the systems described herein.

In some embodiments, there is provided a method of treating ADHD by applying a system as described herein to the skin or mucosa of a subject in need thereof. In some embodiments, the system comprises amphetamine and clonidine, and the system is applied over a period time such as 8 hours, 10 hours, 12 hours, up to about 24 hours, or longer. In some embodiments, the method is effective to achieve therapeutic levels of amphetamine and/or clonidine in the subject during the application period.

As noted above, in some embodiments, the methods achieve different pharamacokinetic profiles for the active agents formulated therein, such as achieving one pharamacokinetic profile for the stimulant therapeutically active agent (e.g., amphetamine or methylphenidate) and a different pharamacokinetic profile for the non-stimulant therapeutically active agent (e.g., clonidine or guanfacine), such as one active agent being delivered at a faster rate and/or over a relatively short period of time as compared to another and/or one active agent exhibiting immediate release and another exhibiting a more sustained release. In specific embodiments, the stimulant therapeutically active agent (e.g., amphetamine or methylphenidate) is delivered at a faster rate and/or over a relatively short period of time and/or exhibits immediate release, while the non-stimulant therapeutically active agent (e.g., clonidine or guanfacine) is delivered at a slower rate and/or over an extended period of time and/or exhibits a more sustained release, or vice versa.

The following specific examples are included as illustrative of the transdermal drug delivery systems and polymer matrices described herein. These examples are in no way intended to limit the scope of the invention. Other aspects of the invention will be apparent to those skilled in the art to which the invention pertains.

### Example 1

A polymer matrix with the following composition is prepared:

| | % w/w in |
|---|---|
| | Finished |
| Ingredient | Dry Product |
| Clonidine | 4 |
| Amphetamine | 15.1 |
| Duro-Tak ® 87-900A | 16.5 |
| Gelva 3087 | 64.4 |
| Total | 100.00 |

The polymer matrix is applied to release liner and dried, a backing material is applied, and transdermal drug delivery systems of a suitable size are prepared.

### Example 2

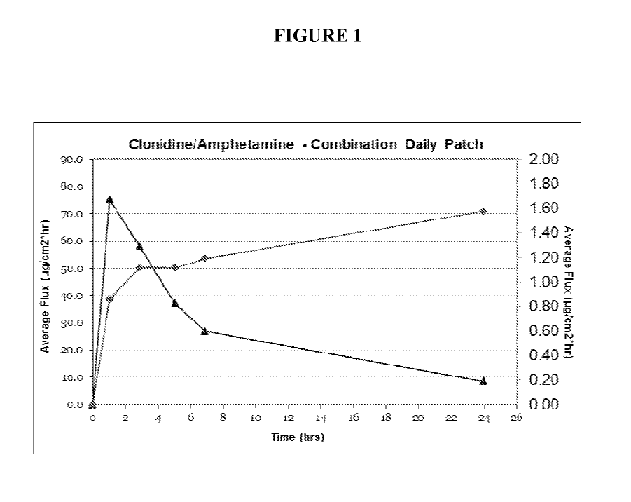

The polymer matrix of Example 1 was used to prepare transdermal drug delivery systems with about 1.05 mg/cm² amphetamine and about 0.28 mg/cm² clonidine. An in vitro flux study was conducted with human cadaver skin using a Franz cell apparatus to determine the flux of each drug from the matrix over a 24-hour period. Results from one sample are shown in FIG. 1.

The results show that the systems according to the invention achieve a sustained flux of clonidine and a flux of amphetamine sufficient to achieve a therapeutic effect. In particular, the results show that the systems according to the invention are capable of delivering the two drugs with two different pharmacokinetic profiles, with the amphetamine delivered at a faster rate over a relatively short period time (e.g., near immediate release) and the clonidine delivered over an extended period of time (e.g., sustained release).

1. A transdermal drug delivery system for the treatment of Attention Deficit Disorder (ADD) or Attention Deficit/Hyperactivity Disorder (ADHD) in the form of a flexible finite system for topical application, comprising a polymer matrix comprising a stimulant therapeutically active agent and a non-stimulant therapeutically active agent.

2. The transdermal drug delivery system of claim 1, wherein the stimulant therapeutically active agent is a central nervous system stimulant.

3. The transdermal drug delivery system of claim 1, wherein the stimulant therapeutically active agent is amphetamine or a pharmaceutically acceptable salt or prodrug thereof.

4. The transdermal drug delivery system of claim 1, wherein the non-stimulant therapeutically active agent is an alpha-2 agonist.

5. The transdermal drug delivery system of claim 4, wherein the non-stimulant therapeutically active agent is clonidine or a pharmaceutically acceptable salt or prodrug thereof.

6. The transdermal drug delivery system of claim 1, wherein the polymer matrix comprises an acrylic polymer.

7. The transdermal drug delivery system of claim 6, wherein the polymer matrix comprises a non-acid functional acrylic polymer.

8. The transdermal drug delivery system of claim 1, wherein the polymer matrix comprises amphetamine, clonidine and a non acid-functional acrylic polymer.

9. The transdermal drug delivery system of claim 1, wherein the polymer matrix comprises a first non-acid functional acrylic polymer and a second non-acid functional acrylic polymer.

10. The transdermal drug delivery system of claim 1, wherein the polymer matrix comprises a non acid-functional acrylic polymer that includes methacrylate monomers and 2-ethylhexyl acrylate monomers.

11. The transdermal drug delivery system of claim 1, wherein the polymer matrix comprises a non acid-functional acrylic polymer that includes methacrylate monomers, 2-ethylhexyl acrylate monomers, and amide-group containing monomers.

12. The transdermal drug delivery system of claim 1, further comprising a backing layer.

13. The transdermal drug delivery system of claim 1, further comprising a release liner.

14. A method of treating ADD or ADHD comprising applying a transdermal drug delivery system according to claim 1 to the skin or mucosa of a subject in need thereof.

15. The method of claim 14, wherein the subject is a human subject.

16. The method of claim 14, wherein the transdermal drug delivery system is applied for a duration of up to 24 hours.

17.-18. (canceled)

19. A method of preparing a transdermal drug delivery system for the treatment of ADHD in the form of a flexible finite system for topical application, comprising preparing a polymer matrix comprising a stimulant therapeutically active agent and a non-stimulant therapeutically active agent.

20. The method of claim 19, wherein the polymer matrix comprises amphetamine, clonidine and a non acid-functional acrylic polymer.
